(19) United States
(12) Patent Application Publication
(10) Pub. No.: US 2009/0286826 A1
(43) Pub. Date: Nov. 19, 2009

(54) PHARMACEUTICAL COMPOSITIONS HAVING CARBOXYVINYL POLYMER AND POVIDONE POLYMER

(76) Inventors: Francisco Javier Galan, Barcelona (ES); Nuria Jimenez, Barcelona (ES); Nuria Carreras, Barcelona (ES)

Correspondence Address: ALCON, IP LEGAL, TB4-8, 6201 SOUTH FREEWAY, FORT WORTH, TX, 76134, US

(21) Appl. No.: 12/464,194

(22) Filed: May 12, 2009

Related U.S. Application Data
(60) Provisional application No. 61/054,196, filed on May 19, 2008.

(51) Int. Cl. A61K 31/4709

(52) U.S. Cl. 514/300

(57) ABSTRACT

The present invention is directed to pharmaceutical compositions, such as ophthalmic gels. The compositions typically include a carboxyvinyl polymer. A povidone polymer is also typically included to stabilize the carboxyvinyl polymer against agents or ingredients (e.g., therapeutic agent) that can otherwise cause instability to the carboxyvinyl polymer.

### CROSS-REFERENCE TO RELATED APPLICATION

The present application claims priority based on U.S. Provisional Patent Application Ser. No. 61/054,196 filed May 19, 2008.

### TECHNICAL FIELD OF THE INVENTION

The present invention is related to pharmaceutical compositions that include carboxyvinyl polymer combined with povidone polymer for providing the compositions with improved compatibility and/or improved physical properties. More particularly, the present invention is related to ophthalmic solutions (e.g., gels) that include therapeutic agent such as moxifloxacin, carboxyvinyl polymer and povidone polymer.

### BACKGROUND OF THE INVENTION

The pharmaceutical industry has continuously sought to produce pharmaceutical vehicles having desirable physical characteristics. Depending upon the type of application (e.g., oral, topical or the like), these characteristics can vary. Many pharmaceutical applications, particularly topical applications of a pharmaceutical composition to the eye (ophthalmic), nose, ear (otic), skin or the like, can be more effective when the pharmaceutical vehicle of the composition provides enhanced viscosity. Gels can be particularly desirable pharmaceutical vehicles when a higher viscosity is desired.

Carboxyvinyl polymer (carbomers) can be used to increase viscosity and/or form gels for pharmaceutical compositions. Carboxyvinyl polymer is particularly desirable for forming aqueous gels. When carboxyvinyl polymer is dispersed in water it will often form a turbid gel. However, if the pH of the gel is greater than the pKa of the carboxyvinyl polymer (e.g., at pH greater than about 6.0±0.5), the turbid gel will typically swell and become clear. In this circumstance, carboxylate groups of the carboxyvinyl polymer are ionized and the negative charges of the carboxylate groups repel each other. In addition, the glass transition temperature of the carboxyvinyl polymer often drops upon exposure of the polymer to water. Under these conditions, the carboxyvinyl polymer gyrates and the radius of gyration can become large and can cause swelling of the polymer up to or greater than 1000 times its original volume. This swelling of the carboxyvinyl polymer can be highly effective in assisting the formation of gels suitable as pharmaceutical vehicles for pharmaceutical compositions.

While carboxyvinyl polymer can be useful for forming desirable gels, the to carboxyvinyl polymer has been shown to be incompatible with numerous ingredients that are often included in pharmaceutical compositions. Examples of such ingredients include, without limitation, high levels of electrolytes, cationic polymers, phenols, strong acids, strong bases, certain amino-functional ingredients (e.g., therapeutic agents), any combination thereof or the like. These incompatibilities can lead to undesirable physical characteristics such as nephelos, polymer degradation, viscosity loss, any combination thereof or the like.

Consequently, it would be particularly desirable to provide a pharmaceutical vehicle and/or pharmaceutical composition that includes carboxyvinyl polymer where that vehicle or composition avoids or reduces one or more of these incompatibilities.

### SUMMARY OF THE INVENTION

The present invention is directed to a pharmaceutical composition. The pharmaceutical composition typically includes that includes carboxyvinyl polymer and povidone polymer. The composition also typically includes a destabilizing agent that normally has a destabilizing effect on the carboxyvinyl polymer, however, that destabilizing effect is typically inhibited by the povidone polymer. The destabilizing agent can include or be substantially entirely or entirely composed of therapeutic agent. In a preferred embodiment, the therapeutic agent (e.g., moxifloxacin) includes one or more amino functional groups and the destabilizing effect is a lack of solubility and/or nephelos caused by the therapeutic agent complexing with the carboxyvinyl polymer to form a therapeutic agent/carboxyvinyl polymer complex. In such embodiment, the povidone polymer assists in solubilizing the therapeutic agent/carboxyvinyl polymer complex. Preferably, the pharmaceutical composition or the aqueous pharmaceutical vehicle thereof is aqueous and/or is a gel. The aqueous pharmaceutical composition can be an ophthalmic composition and preferably has a physiologically compatible pH.

### DETAILED DESCRIPTION OF THE INVENTION

The present invention is predicated upon the provision of a pharmaceutical composition that includes carboxyvinyl polymer and povidone polymer. The pharmaceutical composition will also typically include a destabilizing agent that would normally interact with carboxyvinyl polymer to destabilize the pharmaceutical composition. Advantageously, the povidone polymer can act to remedy this destabilization.

It is contemplated that the pharmaceutical composition may be designed for a variety of applications such as otic, nasal, oral, dermatological or other applications that may be topical or other applications to the skin, ear, nose, mouth or otherwise. The pharmaceutical application has been found particularly desirable as an ophthalmic composition that is topically or otherwise (e.g., intraocularly) administrable to the eye. The composition has also been found to be highly desirable when formulated as a gel and particularly an aqueous gel.

Unless indicated otherwise, all ingredient concentrations are listed as % (w/v).

Carboxyvinyl Polymer

The carboxyvinyl polymer useful in the present invention is preferably ophthalmically, otically and/or nasally acceptable. Typically, such carboxyvinyl polymer will have a network of cross-linked polymer chains. The polymers are characterized as having carboxylic acid functional groups and preferably contain from 2 to 7 carbon atoms per functional group. Prior to cross-linking, the carboxyvinyl polymer useful in the present invention typically has a molecular weight of at least about 50,000, more typically at least about 200,000 and still more typically at least about 400,000 atomic mass units (amu). That molecular weight is typically less than about 6 million, more typically less than about 1 million and still more typically less than about 600,000 amu.

Preferred carbomers or carboxyvinyl polymers are formed of acrylic acid cross-linked with polyalkenyl ethers or divinyl glycol. The polymers are typically polymerized in a solvent such as benzene or ethyl acetate. Ethyl acetate is generally preferred for the present invention since solvent residue can remain with the polymers and ethyl acetate tends to be more biocompatible and/or can have exhibit a relatively lower degree of toxicity relative to some other solvents. The carbomers or carboxyvinyl polymers can be cross-linked with allyl sucrose or allyl penta erythritol.

Preferred carboxyvinyl polymers include water-soluble and water-swellable carbomers, available under the trade name CARBOPOL from the B.F. Goodrich Company. The commercially available polymers Carbopol 934P, 940 and 974P are highly preferred. The amount of carboxyvinyl polymer present in the pharmaceutical composition of the present invention is typically at least about 0.2%, more typically at least about 0.5% even more typically at least about 0.8%. Moreover, the amount of carboxyvinyl polymer present in the pharmaceutical composition of the present invention is typically less than about 10.0%, more is typically less than about 4.0% even more typically less than about 1.2%.

Povidone Polymer

As used herein, povidone polymer is intended to mean is a water-soluble polymer made from two or more N-vinyl pyrrolidone monomers. Povidone polymer used for the present invention is again preferably an ophthalmically, otically and/or nasally acceptable polymer. The povidone polymer can be a mixture of multiple different polyvinyl pyrrolidone polymers and those polymers can be copolymers, homopolymers, otherwise or combination thereof. In one preferred embodiment, however, the povidone polymer is at least 80%, more typically at least 90% and even more typically at least 95% by weight homopolymer of vinyl pyrrolidone, although not required unless otherwise specifically stated. In such an embodiment, it may be possible to employ smaller amounts of povidone polymer to achieved desired stability. In another preferred embodiment, a povidone copolymer is employed as a part or all of the povidone polymer. In such an embodiment, the povidone copolymer may assist in solubilizing therapeutic agent of the pharmaceutical composition particularly wherein a non-povidone monomer of the copolymer is hydrophobic. One example of a suitable copolymer with a hydrophobic non-povidone monomer is vinylpyrrolidone/vinylacetate (VP/VA). Thus, it is contemplated that the povidone polymer can be at least 30%, at least 50% or even at least 90% by weight povidone copolymer, particularly where the povidone polymer includes a hydrophobic monomer such as vinyl acetate.

The povidone polymer preferably has an average molecular weight that is at least about 2000, more particularly at least about 15,000 and still more typically at least about 30,000. The average molecular weight of the povidone polymer is also typically less than about 400,000, more typically less than about 80,000 and still more typically less than about 50,000. Preferred povidone polymer is formed of polyvinyl pyrrolidones such as PVP K60 through PVP K15 (e.g., PVP K30, PVP K25, PVP K15) or more particularly PVP K40 through PVP K20. Example of particularly preferred povidone polymers are sold under the tradenames POVIDONE 29/32, which is commercially available from ISP Technologies (Wayne, N.J., USA) and KOLLIDON 30, which is commercially available from BASF (Ludwigshafen, Germany). The amount of povidone polymer present in the pharmaceutical composition is typically at least about 0.4%, more typically at least about 1.0% even more typically at least about 1.3%. Moreover, the amount of povidone polymer present in the pharmaceutical composition of the present invention is typically less than about 8.0%, more typically less than about 3.0% even more typically less than about 1.7%.

Destabilizing Agent

As used herein, destabilizing agent includes any ingredient or group of ingredients that would normally cause instability (e.g., loss of viscosity, nephelos or the like) of the carboxyvinyl polymer absent the povidone polymer. It is to be understood that the povidone polymer can have varying degrees of effectiveness in stabilizing the carboxyvinyl polymer in the presence of destabilizing agent depending upon the overall pharmaceutical composition.

Generally, the destabilizing agent can include charged or ionic atoms or molecules that provide a negative or positive charge within the pharmaceutical composition particularly when it is an ophthalmic aqueous solution and/or gel.

Such charge or ionic character can normally act to interfere with the swelling of the carboxyvinyl polymer and the povidone polymer can typically act to stop or at least inhibit such interference. Examples of ingredients that can be part or the entirety of the destabilizing agent are, without limitation, high levels of electrolytes, cationic polymers, phenol, strong acids, strong bases, amino-functional actives, combinations thereof or the like.

While it is contemplated that the destabilizing agent can be formed of a variety of different ingredients either alone or in combination, it is preferred that the destabilizing agent be formed partially, entirely or substantially entirely of therapeutic agent. Such therapeutic agent will typically be ionic or charged in the pharmaceutical composition (e.g., solution and/or gel). Moreover, such therapeutic agent can have functional groups such as amino functional groups, which can interact with and cause instability of the carboxyvinyl polymer. It is understood that the functional groups of the therapeutic agent may or may not be charged or ionic within the pharmaceutical composition. It is further understood that the therapeutic agent can include one agent or multiple different agents having one or more of the characteristics discussed.

Examples of preferred therapeutic agents that may be part of the destabilizing agent include, without limitation, aminoglycoside antibiotic (e.g., tobramycin), lysergic acid amide (e.g., cabergoline) or the like. The therapeutic agent can include one or more quinolones (e.g., fluoroquinolones), which may be part of the destabilizing agent. In a preferred embodiment, the therapeutic agent includes one or more amino functional groups that form or would normally form water insoluble complexes with the carboxyvinyl polymer. Such therapeutic agent having one or more amino functional groups can be a quinolone or another agent. Advantageously, the povidone polymer can limit or prevent the formation of such complexes or at least adjust (i.e., increase) the solubility of the therapeutic agent, the carboxyvinyl polymer or both in water or more particularly aqueous gels. One preferred example of a fluoroquinolone that includes at least one amino-functional group is moxifloxacin. Other potentially suitable quinolones (e.g., fluoroquinolones) include, without limitation, ciprofloxacin, levofloxacin, trovafloxacin, enoxacin, garenoxacin, gatifloxacin, germifloxacin, grepafloxacin, lomefloxacin, norfloxacin, ofloxacin, pefloxacin, prulifloxacin, rufloxacin, sitafloxacin, sparfloxacin, temafloxacin, cinoxacin, flumequine, nalidixic acid, oxolinic acid, permidic acid, piromidic acide, rosoxacin, combinations thereof or the like. In a preferred embodiment, the therapeutic agent is at least 90% by weight or entirely quinolone or fluoroquinolone, particularly when that quinolone is any one (e.g., moxifloxacin) or a combination of the aforementioned quinolones.

It should be understood that, while one or more of the therapeutic agents can be included in the destabilizing agent, it is also possible to include therapeutic agent in the pharmaceutical composition where those agents have little or no destabilizing effect upon the carboxyvinyl polymer. Examples of such therapeutic agents include, without limitation, nepafenac and dexamethasone.

The therapeutic agent can be at least 30%, at least 80% or even at least 90% by weight of the destabilizing agent. The therapeutic agent can also be the entirety or the substantial entirety of the destabilizing agent. When included, the amount of destabilizing agent, including the therapeutic agent, can vary widely depending upon the type or types of agents employed. Typically, the destabilizing agent is at least 0.000001 w/v % (weight/volume percent), more typically at least 0.00001 w/v % and even possibly at least 0.0001 w/v % of the pharmaceutical composition. The therapeutic agent is also typically less than 10 w/v % (weight/volume percent), more typically less than 1 w/v % and even possibly less than 0.01 w/v % of the pharmaceutical composition.

The compositions (e.g., gels) of the present invention can include antimicrobial agent. Potential antimicrobial agents include, without limitation, hydrogen peroxide, chlorine containing preservatives such as benzalkonium chloride or others. According to a preferred aspect, however, the pharmaceutical composition of the present invention is entirely or substantially free of any chloride containing preservatives and, particularly, is entirely or substantially free of benzalkonium chloride.

As used herein, the phrase “substantially free of” as it refers to an ingredient of the pharmaceutical composition means that it is contemplated that the pharmaceutical composition can be either entirely devoid of that particular ingredient or includes only a nominal amount of that particular ingredient.

Moreover, the term “substantial” and its derivatives such as “substantially” as those terms modify the term “entire” or its derivatives such as “entirely” means that it is contemplate that all or all except a nominal amount of the particular ingredient being described.

When used, a most preferred antimicrobial agent is polymeric quaternary ammonium compound. The polymeric quaternary ammonium compounds useful in the compositions of the present invention are those which have an antimicrobial effect and which are ophthalmically acceptable. Preferred compounds of this type are described in U.S. Pat. Nos. 3,931,319; 4,027,020; 4,407,791; 4,525,346; 4,836,986; 5,037,647 and 5,300,287; and PCT application WO 91/09523 (Dziabo et al.). The most preferred polymeric ammonium compound is polyquarternium 1, otherwise known as POLYQUAD.RTM. or ONAMERM.RTM. with a number average molecular weight between 2,000 to 30,000. Preferably, the number average molecular weight is between 3,000 to 14,000.

When used, the polymeric quaternary ammonium compounds can be used in the compositions of the present invention in an amount that is greater than about 0.00001 w/v %, more typically greater than about 0.0003 w/v % and even more typically greater than about 0.0007 w/v % of the suspension. Also, when used, the polymeric quaternary ammonium compounds can be less than about 3 w/v %, more typically less than about 0.003 w/v % and even more typically less than about 0.0015 w/v % of the pharmaceutical composition.

The pharmaceutical composition of the present invention can additionally or alternatively include an antimicrobial system such as a borate/polyol complex system, although not required unless otherwise specifically stated. As used herein, is the term “borate” shall refer to boric acid, salts of boric acid, borate derivatives and other pharmaceutically acceptable borates, or combinations thereof. Most suitable are: boric acid, sodium borate, potassium borate, calcium borate, magnesium borate, manganese borate, and other such borate salts. Borate interacts with polyols, such as glycerol, propylene glycol, sorbitol and mannitol, to form borate polyol complexes. The type and ratio of such complexes depends on the number of OH groups of a polyol on adjacent carbon atoms that are not in trans configuration relative to each other. It shall be understood that weight/volume percentages of the ingredients polyol and borate include those amounts whether as part of a complex or not.

As used herein, the term “polyol” includes any compound having at least one hydroxyl group on each of two adjacent carbon atoms that are not in trans configuration relative to each other. The polyols can be linear or cyclic, substituted or unsubstituted, or mixtures thereof, so long as the resultant complex is water soluble and pharmaceutically acceptable. Examples of such compounds include: sugars, sugar alcohols, sugar acids and uronic acids. Preferred polyols are sugars, sugar alcohols and sugar acids, including, but not limited to: mannitol, glycerin, xylitol, sorbitol and propylene glycol.

When used, the borate/polyol complex antimicrobial system (i.e., the borate and polyol together) typically comprise at least 0.05 w/v %, more typically at least 0.5 w/v % and even possibly at least 1 or even at least 1.2 w/v % of the pharmaceutical composition and also typically comprise less than 5 w/v %, more typically less than 2.2 w/v % and even possibly less than 1.5 w/v % of the pharmaceutical composition. The borate to polyol ratio (weight to weight ratio) in the suspension is typically between 1 to 1 and 1 to 10 and more typically is between 1 to 2 and 1 to 4 (e.g., about 1 to 3).

In addition to the ingredients above, it is contemplated that a variety of additional or alternative ingredients may be employed in the pharmaceutical composition of the present invention. Other additional therapeutic agents, antimicrobials or the like may be included in the suspension. Other exemplary ingredients possible for the composition include, without limitation, surfactants, tonicity agents (e.g., NaCl), buffering agents, anti-oxidants, viscosity-modifying agents combinations thereof or the like.

The ingredients described herein may be used in forming various types of pharmaceutical compositions such as ophthalmic, otic, nasal and dermatological compositions, but are particularly useful in forming gels. Examples of such compositions include: ophthalmic pharmaceutical gels, such as topical gels used in the treatment of glaucoma, dry eye, infections, wet and/or dry macular degeneration, conjunctivis, allergies or inflammation; gels for treating contact lenses, such as cleaning products and products for enhancing the ocular comfort of patients wearing contact lenses; and various other types of ophthalmic gels, such as ocular lubricating products, artificial tears, astringents, and so on. The gels may be aqueous or non-aqueous, but will generally be aqueous. Such gels will typically have a viscosity of at least about 250 and even more typically at least about 400 mPa·s at 23° C.

The compositions of the present invention are typically formulated so as to be compatible with the eye and/or other tissues to be treated with the compositions (e.g., gels). The ophthalmic compositions intended for direct application to the eye will be formulated so as to have a pH and tonicity that are compatible with the eye.

The compositions will typically have a pH in the range of 4 to 9, preferably 5.5 to 8.5, and most preferably 5.5 to 8.0. Particularly desired pH ranges are 6.0 to 7.8 and more specifically 6.4 to 7.2. The compositions will typically have an osmolality of 200 to 400 or 450 milliosmoles per kilogram (mOsm/kg), more preferably 240 to 360 mOsm/kg.

As suggested, the pharmaceutical composition can be an ophthalmic, otic or nasal composition that can be topically applied to the eye, ear or nose. The composition can also be applied intravitreal, for example with a needle. Other application of the composition are also considered to be within the scope of the present invention unless otherwise specifically stated.

Advantageously, the use of povidone polymer to stabilize carbomer in the compositions of the present invention can provide significant lowering of nephelos and/or significant increase of viscosity in the compositions of the present invention, particularly aqueous composition. Thus, it is contemplated that the level of nephelos in a pharmaceutical composition of the present invention is at least 5 NTU, more typically at least 10 NTU and even possibly at least 20 NTU less than the level of nephelos in a comparison composition where the comparison composition has exactly the same ingredients as the pharmaceutical composition with the exception that the povidone polymer of the pharmaceutical composition has been replaced with purified water. It is also contemplated that the viscosity in a pharmaceutical composition of the present invention is at least 10 centipoise, more typically at least 100 centipoise, even more typically at least 2000 centipoise and even possibly at least 5000 or even 20,000 centipoise greater than the viscosity of a comparison composition where the comparison composition has exactly the same ingredients as the pharmaceutical composition with the exception that the povidone polymer of the pharmaceutical composition has been replaced with purified water.

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

Other embodiments of the present invention will be apparent to those skilled in the art from consideration of the present specification and practice of the present invention disclosed herein. It is intended that the present specification and examples be considered as exemplary only with a true scope and spirit of the invention being indicated by the following claims and equivalents thereof.

### Example and Experimental Results

Table A below provides a listing of exemplary ingredients suitable for an exemplary preferred formulation of the ophthalmic composition of the present invention and a desired weight/volume percentage for those ingredients.

TABLE A

| Ingredient | w/v percent |
|---|---|
| Moxifloxacin | 0.5 |
| Carboxyvinyl Polymer | 1.0 |
| (Carbomer 974P) | |
| Povidone (PVP K30) | 1.5 |
| Borate (boric acid) | 0.3 |
| Polyol (Sorbitol) | 1.0 |
| Sodium Chloride | 0.35 |
| NaOH and/or HCl | Q.S. to achieve pH = 7.0-7.4 |
| purified water | Q.S. 100 ml |

It is understood that the weight/volume percents in table A can be varied by ±10%, ±20%, ±30%, ±90% of those weight/volume percents or more and that those variances can be specifically used to create ranges for the ingredients of the present invention. For example, an ingredient weight/volume percent of 10% with a variance of ±20% means that the ingredient can have a weight/volume percentage range of 8 to 12 w/v %.

Table B below shows the effects of the povidone on aqueous solutions that include carbomer.

| | Nephelos (NTU) |
|---|---|
| Aqueous Formulation | After autoclave |
| 1.0% Carbomer + 1.0% Sorbitol | 15 |
| 1.0% Carbomer + 0.3% Boric acid | 15 |
| 1% Carbomer + 0.5% Moxifloxacin | 60^{a} |
| 1.0% Carbomer + 0.4% NaCl | 38^{a} |
| 1.0% Carbomer + 1.5% PVP K30 | 11 |
| Carbomer 1.0% | 12 |
| 1.0% Carbomer + 1.0% Sorbitol + 0.3% Boric acid + | 71 |
| 0.4% NaCl + 0.5% Moxifloxacin | |
| 1.0% Carbomer + 1.0% Sorbitol + 0.3% Boric acid + | 6.7 |
| 0.4% NaCl + 0.5% Moxifloxacin + 1.5% PVP K30 | |

^{a}Effect of sodium chloride and moxifloxacin on nephelos.

As can be seen, the povidone can significantly reduce nephelos in the formulations.

We claim:

1. A pharmaceutical composition comprising:
carboxyvinyl polymer and povidone polymer; and
a destabilizing agent that normally has a destabilizing effect on the carboxyvinyl polymer wherein the povidone polymer at least inhibits or decreases the stabilizing effect.

2. A pharmaceutical composition as in claim 1 wherein the destabilizing agent includes a therapeutic agent and the carboxyvinyl polymer and povidone polymer are part of a pharmaceutical vehicle for the therapeutic agent.

3. A pharmaceutical composition as in claim 2 wherein the therapeutic agent includes moxifloxacin.

4. A pharmaceutical composition as in claim 2 wherein the therapeutic agent includes one or more amino functional groups and the destabilizing effect is a lack of solubility caused by the therapeutic agent complexing with the carboxyvinyl polymer to form a therapeutic agent/carboxyvinyl polymer complex and wherein the povidone polymer assists in solubilizing the therapeutic agent/carboxyvinyl polymer complex.

5. A pharmaceutical composition as in claim 1 wherein the pharmaceutical composition or a pharmaceutical vehicle thereof is a gel.

6. A pharmaceutical composition as in claim 1 wherein the pharmaceutical composition is an ophthalmic composition that includes water and has a physiologically compatible pH.

7. A pharmaceutical composition as in claim 1 wherein the composition is contained with a container that emits drops of the composition in a manner suitable for topical application to an eye.

8. A pharmaceutical composition as in claim 1 wherein the destabilizing effect is a loss of viscosity that would otherwise be provided by the carboxyvinyl polymer.

9. A pharmaceutical composition as in claim 1 wherein the destabilizing effect is nephelos caused by interaction of the destabilizing agent with the carboxyvinyl polymer.

10. A pharmaceutical composition as in claim 1 wherein the pharmaceutical composition exhibits a level of nephelos that is at least 10 NTU less than a level of nephelos in a comparison composition where the comparison composition has exactly the same ingredients as the pharmaceutical composition with the exception that the povidone polymer of the pharmaceutical composition has been replaced with purified water.

11. A pharmaceutical composition as in claim 1 wherein the pharmaceutical composition has a viscosity that is at least 2000 centipoise greater than the viscosity of a comparison composition where the comparison composition has exactly the same ingredients as the pharmaceutical composition with the exception that the povidone polymer of the pharmaceutical composition has been replaced with purified water.

12. A pharmaceutical composition comprising:
carboxyvinyl polymer and povidone polymer; and
a destabilizing agent that normally has a destabilizing effect on the carboxyvinyl polymer wherein the povidone polymer at least inhibits or decreases the stabilizing effect;
wherein the destabilizing agent includes a therapeutic agent and the carboxyvinyl polymer and povidone polymer are part of a pharmaceutical vehicle for the therapeutic agent;
wherein the therapeutic agent includes one or more amino functional groups and the destabilizing effect is a lack of solubility caused by the therapeutic agent complexing with the carboxyvinyl polymer to form a therapeutic agent/carboxyvinyl polymer complex and wherein the povidone polymer assists in solubilizing the therapeutic agent/carboxyvinyl polymer complex; and
wherein the pharmaceutical composition is an ophthalmic composition that includes water and has a physiologically compatible pH.

13. A pharmaceutical composition as in claim 12 wherein the therapeutic agent includes moxifloxacin.

14. A pharmaceutical composition as in claim 12 wherein the pharmaceutical composition or a pharmaceutical vehicle thereof is a gel.

15. A pharmaceutical composition as in claim 14 wherein the composition is contained with a container that emits drops of the composition in a manner suitable for topical application to an eye.

16. A pharmaceutical composition as in claim 12 wherein the pharmaceutical composition exhibits a level of nephelos that is at least 10 NTU less than a level of nephelos in a comparison composition where the comparison composition has exactly the same ingredients as the pharmaceutical composition with the exception that the povidone polymer of the pharmaceutical composition has been replaced with purified water.

17. A pharmaceutical composition as in claim 12 wherein the pharmaceutical composition has a viscosity that is at least 2000 centipoise greater than the viscosity of a comparison composition where the comparison composition has exactly the same ingredients as the pharmaceutical composition with the exception that the povidone polymer of the pharmaceutical composition has been replaced with purified water.

18. A pharmaceutical composition as in claim 14 wherein the pharmaceutical composition exhibits a level of nephelos that is at least 10 NTU less than a level of nephelos in a comparison composition where the comparison composition has exactly the same ingredients as the pharmaceutical composition with the exception that the povidone polymer of the pharmaceutical composition has been replaced with purified water.

19. A pharmaceutical composition as in claim 14 wherein the pharmaceutical composition has a viscosity that is at least 2000 centipoise greater than the viscosity of a comparison composition where the comparison composition has exactly the same ingredients as the pharmaceutical composition with the exception that the povidone polymer of the pharmaceutical composition has been replaced with purified water.
